(19) United States
(12) Patent Application Publication
(10) Pub. No.: US 2025/0387129 A1
(43) Pub. Date: Dec. 25, 2025

(54) MEDICAL DEVICE AND PRODUCTION METHOD

(71) Applicant: Acandis GmbH, Pforzheim (DE)

(72) Inventor: Alexander Lange, Karlsruhe (DE)

(21) Appl. No.: 18/871,714

(22) PCT Filed: Jun. 5, 2023
(86) PCT No.: PCT/EP2023/064940

§ 371 Date: Dec. 4, 2024

(30) Foreign Application Priority Data
Jun. 8, 2022 (DE) 10 2022 114 379.1

(51) Int. Cl. A61B 17/221; A61B 17/00; A61F 2/95; B23K 26/38; B23K 103/14

(52) U.S. Cl. CPC A61B 17/221; A61F 2/95; B23K 26/38; A61B 2017/00477; A61B 2017/00526; A61B 2017/2215; A61F 2002/9528; B23K 2103/14

(57) ABSTRACT

A medical device for intravascular treatment, in particular a thrombectomy device, with an at least partially tubular mesh structure which is convertible from a radially compressed state to a radially expanded state and has a multiplicity of cell-forming webs, wherein the mesh structure includes, at a distal end portion, at least two pins which are connected to the webs and have a free end, wherein the pins, in a first longitudinal portion, are pressed flat onto one another at least in parts in order to fix the pin cross-sectional position, and/or the pins, in a second longitudinal portion, are form-fittingly connected to one another, particularly engaging in one another, in order to fix the pin longitudinal position.

### CROSS REFERENCE TO RELATED PATENT APPLICATIONS

This is a U.S. national phase patent application of PCT/EP2023/064940 filed Jun. 5, 2023, which claims the benefit of and priority to DE 10 2022 114 379.1, filed on Jun. 8, 2022, the entire contents of each of which are incorporated herein by reference for all purposes.

### TECHNICAL FIELD

The invention relates to a medical device and a method of production of a medical device.

### BACKGROUND

Medical devices in the form of thrombectomy devices, also known as stent retrievers, are used for the rapid and safe recanalisation of blood vessels. Usually, such thrombectomy devices have a hybrid cell design with a tubular mesh structure in which webs delimit individual cells. Through this, excellent wall apposition and thrombus integration are achieved. For instance, from DE 10 2020 109 158 A1 mentioned above, a thrombectomy device is known which exhibits the aforementioned properties. At a proximal end of the mesh structure, the thrombectomy device has a form-fitting section that is used for connection to a transport wire. On the opposite, distal side, the mesh structure has free web ends, known as pins, which are firmly connected to each other by means of crimp sleeves.

From the prior art it is known that mesh structures of thrombectomy devices are often laser cut from a shape memory material such as nitinol, for example. For this, laser cutting systems are normally used in which the laser cut runs through the longitudinal axis of the tubular shape memory material. This results in a trapezoidal cross-sectional shape of the individual pins. This has the drawback that their position when producing the crimp connection changes in an arbitrary manner. The probability of the crimp connection loosening under light mechanical loading is very high. This leads to the mesh structure becoming distorted and thus considerably more difficult to move in a catheter or becoming stuck at narrow points. In the worst case, the crimp connection could loosen completely so that the crimp sleeve is lost in the blood vessel. A thrombus could therefore no longer be captured properly and thus removed.

Another disadvantage consists in the fact that through the undesirably changed axial position of the pins during the process of loading a catheter, the mesh structure of the thrombectomy device can undergo hogging. This means that the device is not completely compressible and on loading or being pulled back into the catheter after capturing the thrombus, increased frictional resistance between the inserted device and the inner wall of the catheter occurs. In extreme cases it is also possible that during loading or pulling back into the catheter after capturing the thrombus, the thrombectomy device becomes jammed or gets caught in the catheter.

### SUMMARY

The invention is therefore based on the task of providing a medical device, which through an improved structural design exhibits increased functional security and a reliable catheter loading and unloading procedure. The invention is also based on the task of providing a method of producing a medical device.

According to the invention, in relation to the medical device, this task is solved by the subject matter shown and described herein.

More specifically, the task is solved by a medical device for intravascular treatment with an at least partially tubular mesh structure which is convertible from a radially compressed state to a radially expanded state and has a plurality of cell-forming webs. At the distal end section, the mesh structure comprises at least two, more particularly three or more pins which are connected to the webs and have a free end. In a first longitudinal section, the pins are pressed flat onto one another at least in sections in order to fix the pin cross-sectional position.

Additionally, or alternatively, in a second longitudinal section, the pins are form-fittingly connected to one another in order to fix the pin longitudinal position.

Particularly preferably, the medical device is used as a thrombectomy device, more particularly a stent retriever.

The invention has various advantages. Through the flat pressing of the pins in the first longitudinal section, the position of the pins is fixed in the radial direction, that is perpendicularly to the longitudinal extent of the pins. The pins lie flat on each other to that these can be exactly aligned and fixed in their cross-sectional position. This has the advantage that by pressing the pins to each other, e.g. through crimping, adhesion, welding or suchlike, tilting of the pins and thereby distortion or twisting of the tubular mesh structure is prevented.

A further advantage of the invention consists in the fact that the pins are additionally or alternatively fixed in their pin longitudinal position. This takes place through the form-fitting connection of the pins to each other. In other words, the pins are form-fittingly connected to each other in such a way that the longitudinal position of the pins in relation to each other is fixed. This has the advantage that the position of the pins in the longitudinal direction of the pins is secured. Through this, an undesirable relative movement of the pins in the longitudinal direction of the pins is prevented. This means that when introducing the medical device into a catheter, hogging or bulging up of the mesh structure is prevented. Through the fixing of the pin position, the mesh structure is fully compressible, so that introduction of the medical device into the catheter is facilitated. In use, the medical device can be unfolded without delay so that an uniform performance is also achieved. In this way, the invention makes precise radial and axial alignment and fixation of the pins in the distal end section possible, so that increased functional reliability and improved handling are provided.

The pin cross-sectional position is to be understood as the position of the pins perpendicularly to the longitudinal axis of the pins, namely in the radial direction. The pin cross-sectional position is preferably the position of the pins relative to each other perpendicularly to the longitudinal direction of the pins. The pin longitudinal position is to be understood as the position of the pins in the longitudinal direction of the pins, i.e. in the axial direction. The pin longitudinal direction is preferably the position of the pins relative to each other in the longitudinal direction of the pins, which is, in particular, parallel to a longitudinal extent of the pins.

For the form-fitting connection, the pins preferably engage in each other in the second longitudinal section. Preferably the pins engage in each other perpendicularly to their longitudinal direction. Additionally, or alternatively, the pins can engage in each other in the longitudinal direction or be generally connected to each other in a form-fitting manner.

The first longitudinal section and the second longitudinal section define two areas of the pins along their longitudinal extent. Preferably both longitudinal sections border each other in the longitudinal direction of the pins. It is possible that both longitudinal sections of the pins define the entire length of the pins lined up against each other. In other words, the entire length of the pins can be formed by the two longitudinal sections. Preferably the pins are configured in straight lines towards the free end, more particularly in the first and second longitudinal section. The pins preferably run in parallel, especially in the first and second longitudinal sections.

Preferably the pins are adjacent to each other, at least in parts in the first longitudinal section. The pins can be adjacent to each other over the entire length of the first longitudinal section. Alternatively, the pins can be partially adjacent to each other, more particularly over at least a partial length, in the first longitudinal section.

Preferably all pins are the same in shape. Preferably the pins each have identical first longitudinal sections and/or identical second longitudinal sections.

The free ends of the pins preferably form a free distal end of the distal end section of the mesh structure. In the distal end section, the pins preferably form a bundle. The bundle can form a free distal end of the mesh structure. In a preferred form of embodiment, the mesh structure has three pins, which in a first longitudinal section are pressed flat onto each other and/or in the second longitudinal section are form-fittingly connected to each other. Alternatively, the mesh structure can have four, five or more pins.

Preferably the pins form a closed end of the mesh structure. More specifically, as a bundle the pins preferably form a closed distal end of the mesh structure. The pins preferably adjoin a conically tapering area of the tubular mesh structure and close off the mesh structure. In use, the closed distal end of the mesh structure enables better capturing of thrombus fragments and prevents an already captured thrombus in the interior of the mesh structure from sliding out.

It is pointed out that in the context of the patent application, the term “distal” means a side of the medical device turned away from the user, or the remote side of the mesh structure. The term “proximal” means a side of the medical device facing the user, i.e. a side lying closer to the user.

The tubular mesh structure of the medical device is radially expandable and/or compressible. Here, the tubular mesh structure can be transferred from a radially compressed into a radially expanded state by itself, for example use the shape memory effect. The tubular mesh structure of the medical device is thus preferable self-expanding. The mesh structure can be tubular in sections or completely.

In one preferred embodiment, the pins each have at least one radially internal press-on surface with which the pins adjoin each other, wherein the press-on surfaces are aligned in such a way that on exerting a pressing force the pins are fixed in their cross-sectional position. In other words, the pins each have at least one press-on surface which is configured on the inside of the pins. The inside of the pins, more particularly the radially internal pin side, is a respective side of the pins facing each other perpendicularly to the longitudinal direction of the pins, more particularly in the cross-sectional direction. On the pins the press-on surface runs in the longitudinal direction of the pins. Through the press-on surfaces, the pins are in contact with each other. Through the press-on surface, the pins are preferably directly next to each other. In addition, the respective press-on surface is matched to the pins so that the cross-sectional position of the pins in relation to each other is fixed when a pressing force is exerted. Through the internal flat contact, precise alignment of the pins in relation to each other as well as subsequent position-precise fixation of the pins is possible.

In a further preferred form of embodiment, the pins each have on a radially internal pin side, two press-on surfaces converging towards each other for the adjoining of at least one adjacent pin. The two press-on surfaces extend in the longitudinal direction on the pins. In other words, the two press-on surfaces are designed so that the pins on the inside are tapered perpendicularly to the longitudinal direction of the pins. Or expressed in another way, on the internal pin side, the press-on surfaces from press-on diagonals on which in each case one adjacent pin adjoins with one of its press-on surfaces, more particularly press-on diagonals. The arrangement of two internal press-on surfaces has the advantage that the positioning precision when aligning as well as fixing the pins is increased.

The press-on surfaces are preferably straight surface in design. Preferably the press-on surfaces of two adjacent pins adjoining each other are designed to complement each other.

Preferably the pins have at least one radially external force introduction surface, wherein the radially internal press-on surface extends obliquely with regard to the force introduction surface. In other words, the pins each have at least one surface for introducing a pressing force, which is configured on the exterior of the pin. The exterior of the pin is the side of the pins facing away from each other perpendicularly to the longitudinal direction of the pins, more particularly in the cross-sectional direction. Via the force introduction surface, in the fixed state of the pin, a pressing force is introduced into the pins from the outside so that the pressing of at least two adjacent pins onto each other takes place. Through the oblique press-on surface, a partial component of the introduced pressing force is delivered in the direction of the press-on surface, so that at least one further partial component of the pressing force is preferably available for a further press-on surface.

On the pins, the force introduction surface runs in the longitudinal direction of the pins. This can take place at least in sections. Alternatively, the force introduction surface can extend over the entire length of the pins, in particular the first longitudinal section. The force introduction surface preferably has a curvature. In other words, the force introduction surface can be arched. The arch or curvature is preferably convex. It is possible that alternatively, or additionally, the force introduction surface is at least straight in parts, more particularly can at least be partially free of a curvature.

Particularly preferably, in this embodiment the force introduction surface is opposite the two radially internal press-on surfaces, wherein the press-on surfaces are oblique in relation to the force introduction surface. In other words, the radially internal press-on surfaces are at an angle to the force introduction surface. Preferably the two press-on surfaces have the same angle in relation to the force introduction surface these extend in an opposite direction on the inside of the pins. This has the advantage that on introduction of a pressing force into the respective pin, the pressing force is split into two force components of equal magnitude, and these are each deflected in the direction of the two press-on surfaces. Even pressure distribution thus takes place to the two press-on surfaces so that stable fixation of the pins takes place.

In a preferred embodiment, the pins have a narrow surface on their internal side. In other words, the pins each have a radially internal tip that is flattened. Preferably the two radially internal press-on surfaces converge onto the narrow surface. The two press-on surfaces preferably adjoin the narrow surface. The narrow surface preferably extends in the longitudinal direction of the pin, more particularly at least in the first longitudinal section of the pin. The narrow surface is preferably a section of an inner surface (an inner diameter) of a cut, more particularly laser-cut, tubular initial material. This embodiment has the advantage that in their cross-sectional position, due to improve fitting properties, the pins can be positioned more securely.

It is possible that on their inner side, the pins each have a longitudinal edge onto which the two press-on surfaces converge. In other words, the pins each have a radially internal longitudinal edge which is free of a flattened area. Here the two press-on surfaces adjoin each other on the longitudinal edge.

In a preferred embodiment, the pins, more particularly the bundle, have a joint center, that is radially inside, wherein at least one surface normal (FN) of the force introduction surface of the respective pins runs through the joint center. The joint center can form a central area that is provided between the inner sides of the pin. Alternatively, the joint center can be a middle point lying on a common central longitudinal axis of the pins, more particularly the bundle.

In the context of the application, the surface normal FN is an imaginary straight line which at a certain point lies normally on the force introduction surface. The surface normal FN extends perpendicularly to the longitudinal direction of the pins. The surface normal FN corresponds to the effect line of the introduced pressing force in the area of the force introduction surface. The surface normals FN of the force introduction surfaces of all pins preferably intersect in the joint center. Particularly preferably, the common central point forms an intersection at which the surface normals FN of each force introduction surface intersect. In this embodiment, it is advantageous that homogenous force distribution takes place between the pins, so that sliding of the pins in the radial direction is prevented. In other words, through the even force distribution, the respectively predetermined cross-sectional position of the pins is assured, i.e. fixed.

Preferably the pins have at least one joint outer contour which in cross-section approximately corresponds to a regular polygon. The regular polygon can be an equilateral triangle, an equilateral square or an equilateral polygon, for example a star polygon. The regular polygon can be imaginary, i.e. the joint outer contour can be interrupted, so that at least a partial section of the regular polygon is imaginary. It is generally known that the corners of a regular polygon lie on a common circle and thus have the same center angle. More specifically this means that in this form of embodiment the surface normals FN run through a joint center point, so that homogeneous force distribution is possible.

The pins are preferably arranged in a star shape. In other words, as a bundle, the pins preferably have a shar-shaped arrangement. In addition to improved force distribution within the bundle, this allows simpler fixing of the pins to each other, for example by way of a crimp sleeve, an adhesive sleeve and/or welding seams. Due to the star arrangement, stable and robust connection of the pins can take thus take place.

Particularly preferably, the pins comprise at least one ring segment in cross-section. Particularly preferably the pins each form a ring segment in cross-section. In cross-section of the bundle, the pins form a closed annular ring in the circumferential direction. If three pins are provided, the pins preferably each have a ring segment of essentially 120 degrees. If more than three pins are provided, the pins preferably each have a ring segment with the same angular extent. Other, more particularly uneven ring segment divisions are possible. Moreover, alternatively to the ring segment variant, the pins can be circle segment-like in cross-section.

In a preferred embodiment, the medical device comprises at least one connecting element that is arranged on the outside on the pins and presses the pins, particularly in the first longitudinal section, radially onto each other. Particularly preferably the connecting element is a sleeve, more particularly a crimp sleeve, which is crimped with the pings in order to fix them. In this form of embodiment, the sleeve is preferably crimped with the pins in such a way that the sleeve adjoins an inner side on the force introduction surfaces of the pins. This results in homogenous force introduction of a radial retaining force of the sleeve into the pins.

In one area of the force introduction surfaces, the sleeve is deformed in such a way that it introduces a pressing force via the force introduction surfaces into the pins. The inner side of the sleeve can be in point or linear contact with the force introduction surfaces. Preferably the inner side of the sleeve is in flat contact with the force introduction surface. Through this the pins are firmly fixed in position.

Alternatively or additionally, the connecting element can be a sleeve which is adhered to the pins. It is conceivable that alternatively or additionally, the pins are connected to each other in an integrally bonded manner by at least one welding seam, more particularly in the first longitudinal section. Preferably one welding seam for each two adjacent pins is provided. In other words, two adjacent pins are firmly connected to each other by one welding seam. The welding seam can alternatively be a welding point.

Preferably the connecting element had an outer circumference that is essentially circular, more particularly round. In other words, the connecting element has an essentially circular, more particularly round envelope. This has the advantage that the pushability of the medical device, for example, introduction into a catheter or removal from the catheter, is improved.

The connecting element can be circumferentially closed or partially open. More specifically, the sleeve can be circumferentially closed or partially open. The closed sleeve has the advantage that in use the likelihood of losing the sleeve in the blood vessel is reduced. On the other hand, the partially open sleeve has the advantage that emergency opening of the sleeve can take place in the case of overloading. More particularly, it is advantageous if the open sleeve is at least welded onto one of the pins so that in the event of emergency opening, but also of undesired loosening of the open sleeve from the pins, the pin is not lost in the blood vessel. Alternatively or additionally, the sleeve can be adhered to at least one of the pins.

In a preferred embodiment, the pins each have at least one first projection, in particular in the second longitudinal section, and at least one oppositely arranged recess, wherein the first projection of the one pin engages in the recess of the adjacent pin, at least in parts. Through the engagement of the first projection of one pin into the recess of the other pin, precise longitudinal alignment of the pins with each other takes place. Thus, before being pressed onto each other in the first longitudinal section, the pins are precisely positionable in their longitudinal position and fixed in the pressed-on to each other state in their longitudinal position. Through this, the pins are connected to each other in a firmly fixed position in the longitudinal direction. No relative movement of the pins can therefore take place, through which distortion of the mesh structure and consequently dogging, for example during introduction into a catheter, is prevented.

The first projection is preferably an integral part of the respective pin. The first projection preferably extends in the radial direction, i.e. perpendicularly to the longitudinal direction of the pins. It is possible that the pins each have more than one first projection and/or more than one recess. Preferably, the first projection and recess are arranged opposite each on the respective pin in the radial direction. The first projection and the recess are preferably produced by laser cutting. Particularly preferably, a 4-axis laser cutting device is used for this, that makes it possible to perform an eccentric laser cut. More specifically, the laser cutting device can be a 4-axis pipe laser cutting device.

The recess is preferably delimited in the longitudinal direction of the pins by at least two second projections, which each form a longitudinal stop for the first projection of the adjacent pin. In other words, one of the two pins has two projections, which enclose the first projection of an adjacent pin in the longitudinal direction of the pins. The second projections of a pin form longitudinal stops for the first projection of the other pin, so that its movement in both longitudinal directions of the pin is prevented. Preferably, the first projection of one pin engages in the recess of the adjacent pin so that the first projection is in contact with the two projections in the longitudinal direction of the pine, more particularly in a play-free manner.

The first projection and/or the second projections are preferably conical starting from one pin flank. In other words, the projections form teeth that taper towards a tooth tip. This has the advantage that when being pressed onto each other, the pins automatically move into the desired axial position and thus precise longitudinal alignment of the pins with regard to each other takes place.

It is advantageous if the first project and/or the two second projections each have a free end that is rounded. In particular, this has the advantage that in use, the risk of injury of the blood vessel is reduced. Through the rounding, the projections have an atraumatic shape.

In a preferred embodiment, the medical device has at least one cover which envelops the pins in the second longitudinal section, more particularly in the area of the projections. The cover can be formed by at least one adhesive bead. Alternatively or additionally, the cover can comprise at least one adhesive sleeve and/or a coil, more particularly a wire filament. The cover preferably envelopes the absolute distal end of the medical device. This has the advantage that in use a risk of injury to the blood vessel through the medical device is reduced further.

It is possible that in addition to the second longitudinal section, the cover also envelops the first longitudinal section of the pins. The first and/or second longitudinal section of the pins can be partially or full enveloped by the cover. For example, the cover can also envelop the connecting element and/or the at least one welding seam.

Preferably the pins are connected or configured in one piece with webs of the mesh structure. The mesh structure is preferably produced in one piece. This preferably takes place by laser cutting of the mesh structure from a tubular initial material, more particularly a shape memory material.

According to a secondary aspect, the invention relates to a method of producing a medical device, more particularly a thrombectomy device, which comprises a tubular mesh structure which is convertible from a radially compressed state to a radially expanded state and has a plurality of cell-forming webs, wherein at one distal end section, the mesh structure comprises at least two pins connected to the webs, wherein in a first longitudinal section, the pins are pressed flat onto one another at least in parts in order to fix the pin cross-sectional position, and in a second longitudinal section the pins are form-fittingly interconnected, more particularly engage in each other, in order to fix the pin longitudinal position.

In a preferred embodiment, the pins, more particularly the entire mesh structure, are produced by eccentric laser cutting, more particularly using a 4-axis laser cutting device. This has the advantage that on the inner side of the pins, oblique press-on surface can be produced so that optimal positioning of the pins to each other is possible.

With regard to the advantage of the production method, reference is made to the advantages mentioned in connection with the medical device. Moreover, alternatively or additionally, the production method can have individual, or a combination of several features previously stated in in relation to the medical device.

### BRIEF DESCRIPTION OF THE FIGURES

The invention is described below in more detail with reference to the attached drawings. The shown embodiments are examples of how the device according to the invention can be configured.

In these

FIG. 1 shows a partial side view of a medical device according to a preferred example of embodiment;

FIG. 2 shows an enlarged internal view of a pin in the area of a distal end section of a mesh structure of the medical device according to FIG. 1

FIG. 3 shows a perspective view of the distal end section of the mesh structure of the medical device according to FIG. 1 in the crimped state;

FIG. 4 shows a cross-section through a first longitudinal section of the pin of the medical device according to FIG. 1 with a closed crimp sleeve;

FIG. 5 shows a cross-section through a first longitudinal section of the pin of a further medical device according to the invention with an open crimp sleeve;

FIG. 6 shows a perspective view of the medical device according to FIG. 1 in the area of the distal end section of the mesh structure; and

FIG. 7 shows a side view of the medical device in a radially compressed state.

### DESCRIPTION OF AN EMBODIMENT

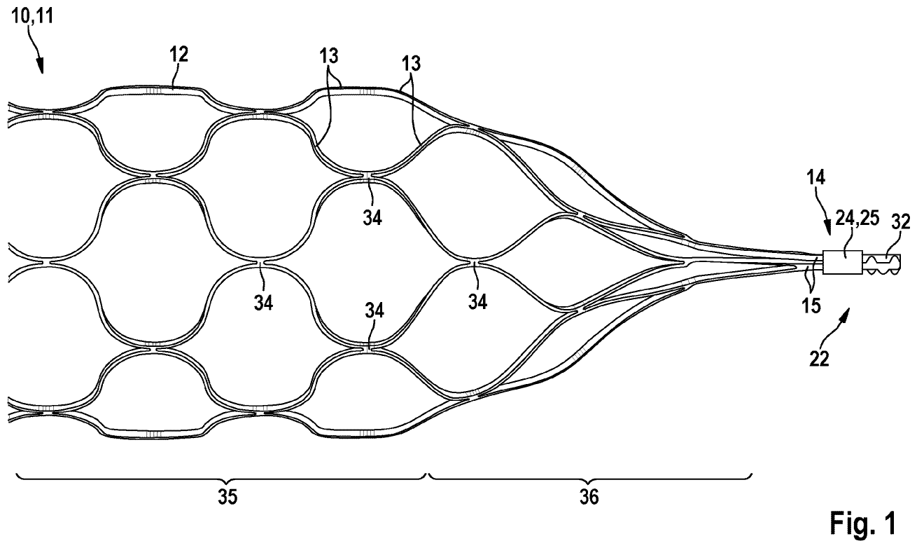

FIG. 1 shows a medical device 10 according to a preferred embodiment according to the invention. The medical device 10 is a thrombectomy device 11 for intravascular treatment. The thrombectomy device 11 is used for removing a thrombus from a blood vessel. The medical device 10 can also be described as a stent retriever. In the following description the medical device 10 is in general called a thrombectomy device 11.

The thrombectomy device 11 comprises a mesh structure 12 which is formed of interconnected webs 13. The webs 13 are connected to each other at web connectors 34, wherein the web connectors 34 each connect four webs 13 to each other. Here, the web connectors 34 are preferably Y connectors. It is possible that web connectors 34 are provided which each connect three webs 13 to each other. More particularly, web connectors 34 can be provided that each (only) connect two webs 13 to each other. This can be the case, for example, with short end cells. Preferably the mesh structure 12 designed to be rotationally symmetrical, a least in parts. The mesh structure 12 is radially compressible as well as radially expandable on its own.

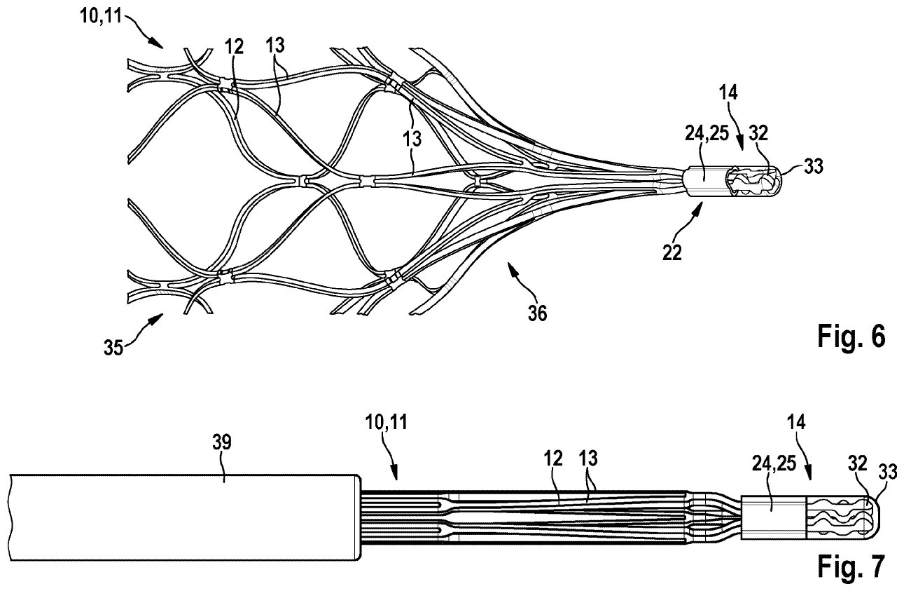

As can be seen in FIGS. 1 and 6, the mesh structure 12 is partially tubular in design. In other words, the mesh structure 12 has a tubular area 35. The mesh structure 12 has a central longitudinal axis. The mesh structure 12 also comprises a conical area 36 and adjoins the tubular area 35. The mesh structure 12 has a, not shown, proximal end section and a distal end section 14. The distal end section 14 is taken to mean an end of the mesh structure 12 facing away from a user of the thrombectomy device 11. The proximal end section is taken to mean an end of the mesh structure 12 facing the user of the thrombectomy device 11.

The conical area 36 converges from the tubular area 35 onto the distal end section 14. The distal end section 14 forms a closed end of the mesh structure 12. In the distal end section 14, the mesh structure 12 has several pins 15 which in the distal direction have a free end. The pins 15 will be discussed in more detail later.

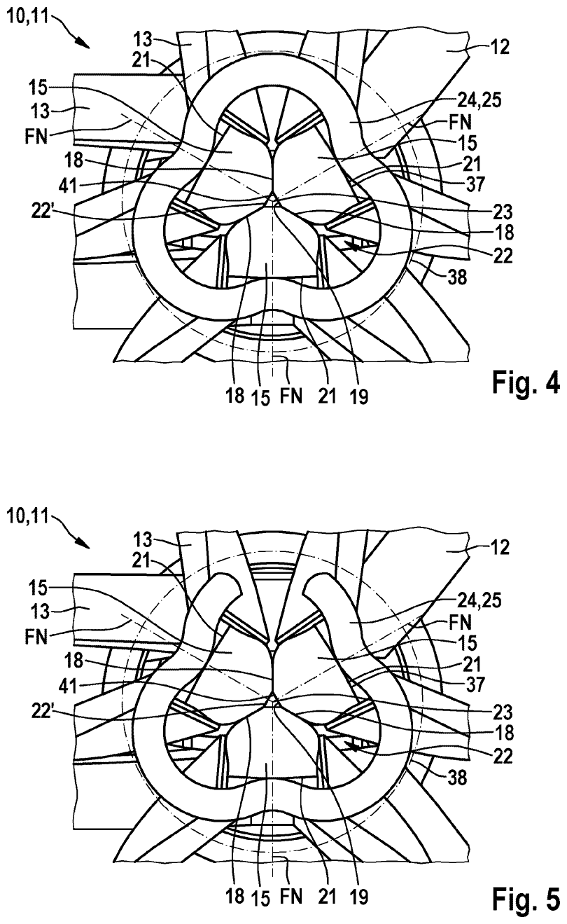

The mesh structure 12 is laser-cut from a pipe material. The pipe material is a shape memory material. More specifically, the mesh structure 12 is cut by a 4-axis laser cutting device which makes an eccentric laser cut possible. In other words, the mesh structure 12 is at least partially cut by an eccentric laser cut. This means that during laser cutting, the laser beam does not cut the central longitudinal axis of the pipe material. Through this, the webs 13 and/or the pins 15 can have a cross-section deviating from a standard trapezoidal cross-section. This can be clearly seen in FIGS. 4 and 5. Other geometries of the mesh structure 12 are possible. In addition to laser cutting, other production methods are possible.

As described above, in the distal end section 14, the mesh structure 12 has several pins 15 with a free end. According to FIGS. 3, 4 and 5, it can be clearly seen that the mesh structure 12 has three pins 15 in total. Alternatively, the mesh structure 12 can also have two or more than three pins 15.

The pins 15 form a free, distal end 32 of the mesh structure 12. The free end 32 of the mesh structure 12 is radially closed, i.e. perpendicularly to the central longitudinal axis. In other words, the mesh structure 12 has a closed distal end 32. In use, this enables better capturing of thrombus fragments and prevents a thrombus already taken up in the interior of the mesh structure 12 from sliding out again.

In the distal end section 14, the pins 15 form a bundle 22. In other words, in the distal end section 14, to form the closed end, the pins 15 are radially bundled. In the distal end section 14, the pins 15 extend at least partially in a straight line. The pins 15 run in parallel. The pins 15 extend along the central longitudinal axis of the mesh structure 12. Expressed otherwise, the pins 15 extend in the longitudinal direction of the mesh structure 12,

The pins 15 are part of the mesh structure 12. More specifically, the pins 15 are produced in one piece, more particularly monolithically, with the webs 13 of the mesh structure 12. As shown in FIGS. 1, 3, 6, and 7, the pins 15 are connected with the webs 13 of the conical area 36 of the mesh structure 12.

The pins 15 have a first longitudinal section 16 and a second longitudinal section 17 which adjoins the first longitudinal section 16. In first longitudinal section 16, the pins 15 for fixing the pin cross-sectional position are radially pressed flat on to each other on the inside. In the second longitudinal section 17, the pins 15 for the fixing pin longitudinal position are connected to each other in a form-fitting manner.

In order to press the pins onto each other or fix them, the thrombectomy device 1 comprises a connecting element 24. The connecting element 24 is preferably radiographically visible. The connecting element 24 has an essentially round outer circumference 38. According to FIGS. 4 and 5, the connecting element 24 is a crimp sleeve 25. The crimp sleeve 25 can be closed in the circumferential direction (FIG. 4) or be partially open in the circumferential direction (FIG. 5). In other words, the crimp sleeve 25 according to FIG. 5 can have a longitudinal slit. Or, expressed otherwise, the crimp sleeve 25 is longitudinally divided in design.

The crimp sleeve 25 is arranged outside on the pins 15 in the first longitudinal section 16. The crimp sleeve 25 is crimped with the pins 15 for radial fixation. In addition to fixing the pins, the crimp sleeve 25 acts as an X-ray marker.

Alternatively or additionally, it is possible that the connecting element 24 has an adhesive sleeve, which is arranged outside on the pins 15 and fixes the pins 15 in their cross-sectional position. Here, the pins 15 are adhered to each other for fixation. Alternatively or additionally, the pins 15 can be welded to each other. For example, according to FIGS. 4 and 5, the crimp sleeves 25 can also be welded to at least one of the pins 15. It is possible that the pins 15 are connected to each other by at least one welding seam. Furthermore, it is possible that two adjacent pins 15 are connected to each other by a weld more particularly by at least one welding seam or at least one welding bead.

As described above, the pins 15 in the first longitudinal section 16 are pressed flat onto each other. For this, the pins 15 each have two press-on surfaces 18 on a radially internal pins side 19, more particularly inner side. The press-on surfaces 18 extend along the longitudinal direction of the pins in the first longitudinal section 16. It is possible that both press-on surfaces 18 also extend in the second longitudinal section 17 of the pins 15. The press-on surfaces 18 are designed to converge towards each other on the radially internal pin side 19. The press-on surfaces 18 are each for the contacting of an adjacent pin 15.

As can be seen in FIGS. 4 and 5, two adjacent pins 15 adjoin each other each with one press-on surface 18. In other words, two adjacent pins 15 are in flat contact through the press-on surfaces 18. The press-on surfaces 18 have a flat surface. The two press-on surfaces 18 of the respective pins 15 extend in parallel in the longitudinal direction of the pins. The press-on surfaces 18 of the pins 15 are configured so that during the introduction of a radial pressing force of the crimp sleeve 15, the pins 15 are fixed in their cross-sectional position.

The pins 15 also each have a radially external pins side 37, more particularly outer side, on which the crimp sleeve 25 lies. In order to introduce the pressing force or retaining force of the crimp sleeve 25 into the respective pin 15, the pins 15 have a radially external force introduction surface 21 which extends in longitudinal direction of the pins in the first longitudinal section 16. In other words, the force introduction surface 21 is configured on the radially external pin side 37. The force introduction surface 21 is arranged opposite the two radially internal press-on surfaces 18. The two press-on surfaces 18 are oblique in relation to the force introduction surface 21.

The radially external pin side 37 of the pins 15 is the side of the pins 15 facing away from each other perpendicularly to the longitudinal direction of the pins 15, more particularly perpendicularly to the central longitudinal axis. In the fixed state of the pins 15, via the force introduction surface 21, a pressing force of the crimp sleeve 25 is introduced into the pins 15 from outside, so that the press-on surfaces 18 of two adjacent pins 15 are pressed onto each other. Due to the oblique press-on surfaces 18, a partial component, more particularly partial components of equal magnitude, is/are diverted in the direction of the press-on surfaces so that even distribution of the pressing force onto both press-on surfaces 18 takes place.

The force introduction surface 21 extends over the entire length of the first longitudinal section 16 of the pins 15. Alternatively, this can be in parts. The force introduction surface 21 has a curvature. In other words, the force introduction surface 21 is arched in design. As can be seen in FIGS. 4 and 5, this arching or curvature is convex.

FIGS. 4 and 5 also show that the pins 15 are arranged in a star shape. It can also be seen that the pins 15 are ring segment-shaped in cross-section. In other words, the pins 15 each have a ring segment in cross-section. The ring segments cover an angular area of approximately 120 degrees in the circumferential direction.

Here, the pins 15 have a joint center 22′ that is radially inside. The central longitudinal axis of the mesh structure 12 runs through the joint center 22′. The joint center 22′ is formed by a central area that is provided between the radially internal pin sides 19. According to FIGS. 4 and 5, the joint center 22′ is a free space that is formed between the radially internal pin sides 19 of the pins 15. On their radially internal pin sides 19, the pins 15 have a flattened tip 41. In other words, on their radially internal pin sides 19, the pins 15 each have a narrow surface. The two radially internal press-on surface 18 converge on the narrow surface. The two press-on surfaces 18 radially adjoin the narrow surface. In the longitudinal direction of the pins, the narrow surface extends at least in the first pin longitudinal section 16. The narrow surface is a section of an inner diameter of a cut, more particularly laser-cut, tubular initial material. Because of the radially internally flattened tips 41 of the pins 15, the joint center 22′ is essentially triangular in cross-section.

The pins 15 are arranged with one another in such a way that a surface normal FN of the force introduction surfaces 21 of the relative pin 15 runs through the joint center 22′. The surface normal FN is the imaginary straight line that is normal to the force introduction surface 21. The surface normal FN corresponds to the effect line of the introduced pressing force in the area of the force introduction surface 21. In the specific case, which is the ideal case, the surface normals FN of the force introduction surfaces 21 of the pins 15 intersect in a common center point 23 which lies on the central longitudinal axis of the mesh structure 12. It is possible that the surface normals FN of the force introduction surfaces 21 of the pins 15 run approximately, i.e. at a small distance, around the common central point 23. This represents the real case.

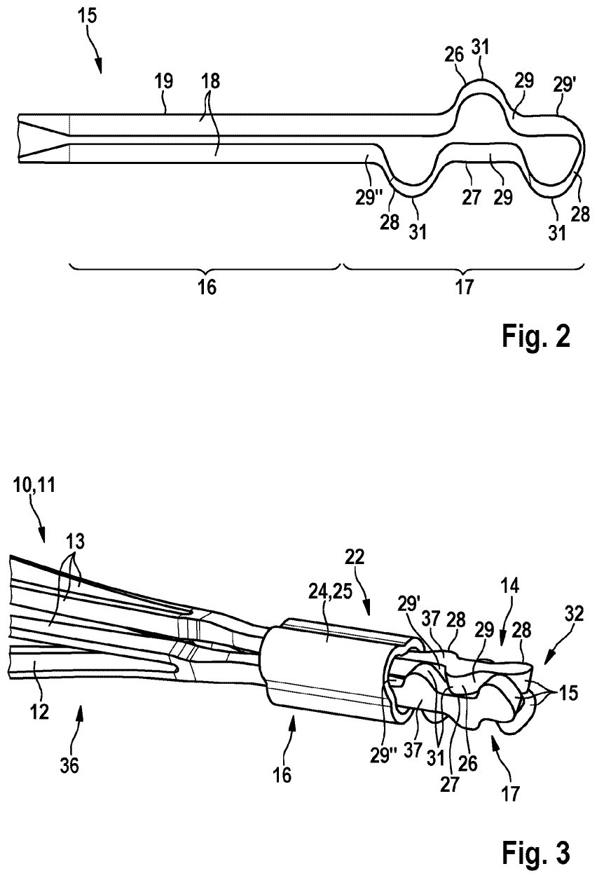

As described above, the pins 15 are form-fittingly interconnected in the second longitudinal section 17. According to FIGS. 2 and 3 it is shown that the pins 15 in the second longitudinal section 17 each have a first projection 26 and an oppositely arranged recess 27. The first projection 26 is arranged on a pin flank 29′ of the respective pin 15. The recess 27 is formed on a second pin flank 29″ which is opposite the first pin flank 29′. The first projection 26 of the respective pin 15 engages in the recess 27 of the respective adjacent pin 15. In other words, the first projection 26 of the respective pin 15 projects into the recess 27 of the respective adjacent pin 15. The recess 27 forms a receptacle for the first projection 26 of the adjacent pin 15. In the longitudinal direction of the pin, the recess 27 is delimited by two second projections 28. The second projections 28 each form a longitudinal stop for the engaging first projection 26 of the adjacent pin 15. Through this, the longitudinal position of the pins 15 with regard to each other is fixed.

Starting from the respective pin flank 29′, 29″, the first projection 26 and the second projections 28 of the pins 15 are conical in design. The projections 26, 28 are tooth-like in design. The first projection 26 and the second projections 28 form toothing to fix the longitudinal position of the pins 15. The first projection 26 and the second projections 28 extend in opposite directions on the respective pin 15. The projections 26, 28 extend in a circumferential direction of the bundle 22 of pins 15. The projections 26, 28 of the pins 15 each have a free end 31 that is rounded. In other words, the projections 26, 28 have a round tip and thus an atraumatic shape.

As shown in FIGS. 6 and 7, the thrombectomy device 1 comprises a cover 33 that envelopes the pins 15 in the second longitudinal section 17. Specifically, the cover 33 envelopes the projections 26, 28 and the recesses 27. The cover 33 can be made of an adhesive bead. Alternatively, the cover 33 can be pushed onto the free end of the pin 15 and adhered thereto. The cover 33 and the crimp sleeve 25 essentially have an identical outer circumference 38.

FIG. 7 also shows the thrombectomy device 11 when being pushed into or out of a catheter 39. The mesh structure 12 of the thrombectomy device 11 is in a radially compressed state. From FIG. 7 it can be clearly seen that through the exact radial and axial position of the pins 15 with regard to each other, problem-free movement of the compressed thrombectomy device 11 into the catheter and out of the catheter is made possible.

### LIST OF REFERENCE NUMBERS

10 Medical device
11 Thrombectomy device
12 Tubular mesh structure
13 Cell-forming web
14 Distal end section of the mesh structure
15 Pins
16 First longitudinal section of the pins
17 Second longitudinal section of the pins
18 Press-on surface
19 Radially internal pin side
21 Force introduction surface
22 Bundel
22′ Joint center
23 Common central point
24 Connecting element
25 Crimp sleeve
26 First projection
27 Recess
28 Second projections
29′ First pin flank
29″ Second pin flank
31 Free end of the projections
32 Closed end
33 Cover
34 Web connector
35 Tubular area
36 Conical area
37 Radially external pin side
38 Outer circumference
39 Catheter
41 Flattened tip
FN Surface normal

1-21. (canceled)

22. A medical device for intravascular treatment with an at least partially tubular mesh structure which is convertible from a radially compressed state to a radially expanded state and has a plurality of cell-forming webs, wherein the mesh structure comprises, at a distal section, at least two pins which are connected to the webs and have a free end, wherein the pins, in a first longitudinal section are pressed flat onto one another at least in sections in order to fix a cross-sectional position of the pins, and/or the pins in a second longitudinal section are form-fittingly connected to one another in order to fix a longitudinal position of the pins.

23. The device according to claim 22, wherein the pins each have at least one radially internal press-on surface, with which the pins adjoin each other, wherein the at least one radially internal press-on surface is aligned in such a way that on introducing a pressing force, the pins (15) are fixed in their cross-sectional position.

24. The device according to claim 22, wherein the pins each have on a radially internal pin side and two press-on surfaces converging towards each other for contacting at least one adjacent ones of the pins.

25. The device according to claim 23, wherein the pins further comprise at least one radially external force introduction surface, wherein the at least one radially internal press-on surface is oblique in relation to the at least one radially external force introduction surface.

26. The device according to claim 25, wherein the pins further comprise a joint center which is radially inside, wherein at least one surface normal FN of the at least one radially external force introduction surface of a relative one of the pins runs through the joint center.

27. The device according to claim 22, wherein the pins have at least one common outer contour, which in cross-section approximately corresponds to a regular polygon.

28. The device according to claim 22, wherein the pins are arranged in a star shape with each other.

29. The device according to claim 22, wherein the pins are ring or circle segment-shaped in cross-section.

30. The device according to claim 22, wherein at least one connecting element that is arranged externally on the pins and radially presses the pins onto each other.

31. The device according to claim 30, wherein the connecting element has an outer circumference which is essentially circular.

32. The device according to claim 30, wherein the connecting element is circumferentially closed or partially open.

33. The device according to claim 22, wherein the pins are connected to each other in a bonded manner by at least one welding seam.

34. The device according to claim 22, wherein the pins each have at least one first projection and at least one oppositely arranged recess, wherein the first projection of one of the pins at least partially engages in the recess of an adjacent one of the pins.

35. The device according to claim 34, wherein in a longitudinal direction of one of the pins, the recess is delimited by at least two second projections which each form a longitudinal stop for the first projection.

36. The device according to claim 35, wherein starting from one pin flank, the first projection and/or the second projections is/are conical in design.

37. The device according to claim 35 wherein the first projection and/or the second projections each have a free end that is rounded.

38. The device according to claim 35, wherein at least one cover is provided, that envelopes the pins in the second longitudinal section.

39. The device according to claim 22, wherein the pins form a closed end of the mesh structure.

40. The device according to claim 22, wherein the pins are connected in one piece with the webs of the mesh structure.

41. A method of producing a medical device, with a tubular mesh structure which is convertible from a radially compressed state to a radially expanded state and has a plurality of cell-forming webs, wherein the mesh structure further comprises, at a distal section, at least two pins which are connected to the webs wherein the pins, in a first longitudinal section are pressed flat onto one another at least in sections in order to fix a cross-sectional position of the pins, and the pins in a second longitudinal section are form-fittingly connected to one another in order to fix a longitudinal position of the pins.

42. The method according to claim 41, wherein the pins are produced by eccentric laser cutting.
